(19) United States
(12) Patent Application Publication
(10) Pub. No.: US 2006/0013834 A1
(43) Pub. Date: Jan. 19, 2006

(54) Room temperature stable aqueous liquid pharmaceutical composition

(75) Inventors: Emmett Clemente, Manchester, MA (US); Bhiku Patel, Chandler, AZ (US); Kuljit Bhatia, Chandler, AZ (US); Frank L. Sorgi, Sonoma, CA (US); Emil D. Kakkis, Novato, CA (US)

Correspondence Address: MARSHALL, GERSTEIN & BORUN LLP, 233 S. WACKER DRIVE, SUITE 6300, SEARS TOWER, CHICAGO, IL, 60606, US

(73) Assignee: MEDICIS PEDIATRICS, INC., Scottsdale, AZ (US)

(21) Appl. No.: 11/066,113

(22) Filed: Feb. 25, 2005

Related U.S. Application Data
(63) Continuation-in-part of application No. 10/786,435, filed on Feb. 24, 2004.

(51) Int. Cl. A61K 9/00

(52) U.S. Cl. 424/400

(57) ABSTRACT

A liquid pharmaceutical composition is contemplated that comprises a pharmaceutically effective amount of a drug dissolved or dispersed in an aqueous medium. The aqueous medium consists essentially of water, about 3% to about 10% w/v polyvinylpyrrolidone, about 60% to about 75% w/v of C₃-C₆ polyol that includes more than 55% w/v of a non-reducing disaccharide, trisaccharide or tetrasaccharide such as sucrose, optionally about 0.01% to about 0.5% w/v of a glycyrrhetic acid, glycyrrhizinate derivative or salt thereof, and one or more flavorants, and preferably includes one or more preservatives. The liquid composition is room temperature stable, and may have a pleasant taste.

The present application is a continuation-in-part application of U.S. Ser. No. 10/786,435 filed Feb. 25, 2004. The entire text of the aforementioned application is incorporated herein by reference.

### TECHNICAL FIELD

The present invention relates to a liquid drug composition, and more particularly to a room temperature-stable, microbially-protected, pleasant-tasting (due to the use of debittering agents and taste-masking technologies) aqueous liquid pharmaceutical composition of a steroidal drug, specifically, prednisolone sodium phosphate.

### BACKGROUND ART

Many useful, effective drugs have a bitter taste or are unstable when dissolved in liquid form. Bitter-tasting or potentially unstable drugs are consequently usually formulated for oral administration as coated tablets or capsules or as a powder or liquid within a capsule so that the bitter tasting medicament does not contact the tongue during oral administration.

Although formulations of the bitter-tasting drug into a coated tablet, capsule or liquid within a capsule alleviates the problem of offensive taste for most of the adult population that uses those drugs, many adults and children have difficulty swallowing oral solid dosage forms (tablets and capsules). U.S. Pat. No. 5,455,049 illustrates one technique that was successful in overcoming the bitter taste problem associated with orally-administered terfenadine.

U.S. Pat. No. 5,763,449 and No. 5,962,461 describe a pleasant-tasting aqueous liquid composition that can contain prednisolone sodium phosphate (PSP) as the active ingredient. Work with a PSP formulation prepared in accordance with the above-noted patent provided a commercially useful pharmaceutical composition. However, the pharmaceutical composition described in the '449 patent required refrigeration prior to use to maintain stability of the PSP and to protect the composition from microbial attack.

Thus, there remains a need for pleasant-tasting orally-administerable formulations of bitter tasting drugs such as PSP, which are stable at ambient, room temperature and contain preservatives which impart the necessary antimicrobial activity required for a pharmaceutically-viable product.

### BRIEF SUMMARY OF THE INVENTION

The disclosure that follows illustrates specific resolution to the problems of bitter taste and oral administration of PSP. These PSP compositions are particularly suitable to adults and children that have difficulty swallowing or cannot swallow tablets, capsules and the like. Preferably, the drug is formulated as an aqueous composition that can be stored at ambient room temperature while providing stability to the active ingredient and minimizing microbial attack at an acceptable level (i.e., conforms to the USP antimicrobial effectiveness test).

The present invention provides disclosure of a liquid pharmaceutical composition. That composition is a liquid pharmaceutical composition comprising a pharmaceutically effective amount of a steroid or other bitter-tasting drug such as prednisolone sodium phosphate (PSP) dissolved or dispersed in an aqueous medium. The aqueous medium comprises water, optionally about 3% to about 10% weight/volume (% w/v) polyvinylpyrrolidone (PVP), about 60% to about 75% w/v of a C₃-C₆ polyol that includes at least 55% w/v of the total composition of a non-reducing disaccharide or trisaccharide such as sucrose, and optionally, about 0.01 to about 5.0% w/v, and more preferably, 0.01 to about 0.5% w/v of glycyrrhetic acid, a glycyrrhizinate derivative such as an ester, amide or thioester of glycyrrhetic acid, or a salt thereof and one or more flavorants. The liquid composition contemplated herein is transparent and may have a pleasant taste when orally administered; i.e., it is substantially free from having a bitter taste that would otherwise be associated with the bitter-tasting active ingredient such as PSP. A contemplated composition is designed for storage at ambient room temperature; i.e., typically at about 20-35° C.

In some embodiments, the drug is present in an amount of less than about 0.5% w/v of the total weight of the composition. In other embodiments, the composition is such that the C₃-C₆ polyol is present as a mixture of C₃ polyol and C₆ polyol, that the mixture of C₃ polyol and C₆ polyol being present in an amount of about 60% to about 75% w/v, and that the weight ratio of the C₃ polyol to the C₆ polyol be about 1:6 to about 1:70. The polyvinylpyrolidone (PVP) is preferably present at about 3% to about 7% w/v.

The invention contemplates compositions that are stable with respect to the active pharmaceutical ingredient (API) and can be stored at ambient room temperature without degradation of the API. In one embodiment, the drug interacts with ammonium glycyrrhizinate (a glycyrrhetic acid derivative), sucrose and preservatives to enhance the stability of the formulation comprising the drug.

The invention further contemplates compositions that are well-preserved and less prone to microbial contamination as compared to prior art preparations of the same.

Advantageously, the contemplated compositions have a pleasant taste that permits them to be administered to children without the usually observed reluctance of children to take the bitter tasting drug. In addition, the compositions are particularly useful for administration to children and the elderly as the compositions are substantially free of ethanol or other alcohols.

Further, exemplary contemplated compositions are homogeneously dispersed and non-settling so that one need not resuspend the medication within the composition prior to each administration and each dose contains a desired amount of the medicament.

Still further benefits and advantages of the invention will be apparent to those skilled in the art from the disclosure that follows.

### DETAILED DESCRIPTION OF THE INVENTION

Oral administration of pharmaceutically active agents is a particularly convenient form of administration of a medicament. Active agents can be given to the patient orally as liquid solutions, oral dispersions (emulsions, suspensions, colloids, liposomes, micelles and the like) or in solid form such as capsules or tablets. Infants, children, the elderly and many other persons often have difficulty in swallowing tablets or bitter-tasting solutions.

The present invention is designed to alleviate these problems and provides orally-administrable formulations of bitter-tasting and/or unstable medicaments which are preferably stable at room temperature. The compositions of the present invention may readily be prepared using any bitter-tasting drug. Exemplary such drugs include, but are not limited to acetaminophen, terfenadine, guaifenesin, dexamethasone, triamcinolone, trimethoprim, prednisolone, ibuprofen, prednisolone sodium phosphate, methacholine, neostigmine, epinephrine, albuterol, pseudoephedrine hydrochloride, diphenhydramine, chlorpheniramine maleate, phenothiazine, chlorpromazine, chlordiazepoxide, amitriptyline, barbiturates, steroids, prednisone, diphenylhydantoin, caffeine, morphine, demerol, codeine, diphenoxylate, opiate derivatives, benzodiazepines, lidocaine, antidepressants, opioids, prostaglandine analogs (e.g. misoprostol), glucocorticosteroids, cytostatics, methotrexate, cimetidine, ranitidine, pantoprazole, omeprazole, lansoprazole, furosemide, penicillamine, sulfasalazine, auranorfin, salicylic acid, fluticasone propionate, sulfonamides, salbutamol, formoterol, salmeterol, terbutaline, beclometasone dipropionate, flunisolide, budesonide, chloroquine, pregnanes, desogestrel, allylestrenol, ethylestrenol, lynestrenol, carpipramine, clomipramine, desipramine, imipramine, imipraminoxide, imipramine pamoate, lofepramine, metapramine, opipramol, quinupramine, trimipramine, amitriptyline, chlordiazepoxide, medazepram, pridinol, perphenazine, amitriptylinoxide, butriptyline, cyclobenzaprine, demexiptiline, nortriptyline, diazepam, perphenazine, fluphenazine, flupentixol, noxiptilin, protriptyline, adinazolam, amoxapine, dibenzepin, dimetacrine, dosulepin, dothiepin, fluacizine, fluoracyzine, toracizin, iprindole, maprotiline, melitracen, pizotyline, propizepine, tianeptine, 5-hydroxytryptophan, ademetionine, amfebutamone, amfebutamone hydrochloride, amineptine, amineptine hydrochloride, amisulpride, fluoxetine hydrochloride, fluoxetine, hypericin, sertraline hydrochloride, sertraline, trimipramine maleate, citalopram, citalopram hydrobromide, clomipramine chloride, d-phenylalanine, demexiptiline, demexiptiline hydrochloride, dimethacrine tartrate, dothiepin, dothiepin hydrochloride, doxepin, fluphenazine hydrochloride, fluvoxamine, fluvoxamine hydrogen maleate, fluvoxamine maleate, indalpine, isocarboxazide, 1-tryptophan, lofepramine, maprotiline, maprotiline hydrochloride, maprotiline mesilate, medifoxamine, metaprimine fumarate, mianserin, moclobemide, nitroxazepine hydrochloride, nomifensine, nomifensine maleate, nomifensin hydrogenmaleat, oxitriptan, paroxetine, paraoxetine hydrochloride, pheneizine, pheneizine sulfate, piracetam, pirlindole, pivagabine, prolintane hydrochloride, propizepine hydrochloride, protriptyline hydrochloride, quinupramine, remoxipride hydrochloride, setiptiline maleate, tianeptine sodium, trazodone hydrochloride, venlafaxine hydrochloride, maprotiline, toloxatone, tranylcypromine, trazodone, trazodone hydrochloride, viloxazine, viloxazine hydrochloride, zimelidine, zimelidine dihydrochloride, alfentanil, allylprodine, alphaprodine, anileridine, benzylmorphine, bezitramide, buprenorphine, butorphanol, clonitazene, cyclazocine, desomorphine, dextromoramide, dezocine, diampromide, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene fentanyl, heroin, hydrocondone, hydromorphone, hydroxypethidine, isomethadone, dextropropoxyphene, ketobemidone, levallorphan, levorphanol, levophenacylmorphan, lofentanil, meperidine, meptazinol, metazocine, methadone, metopon, morphine, myrophine, nalbuphine, narceine, nicormorphine, norlevorphanol, normethadone, naiorphine, normorphine, norpipanone, opium, oxycodone, oxymorphone, papaveretum, pentazocine, phenadoxone, phenomorphan, phenazocine, phenoperidine, piminodine, piritramide, propheptazine, promedol, properidine, propiram, propoxyphene, sufentanil, tilidine, tramadol, nutritional supplements, antacids, analgesics, anti-inflammatories, antipyretics, antibiotics, antimicrobials, laxatives, anorexics, antihistamines, antiasthmatics, antidiuretics, antiflatuents, antimigraine agents, antispasmodics, sedatives, antihyperactives, antihypertensives, tranquilizers, decongestants, beta blockers, vitamin preparations, minerals and penicillins.

In particular exemplary embodiments, the bitter-tasting drug is prednisolone sodium phosphate (PSP) or prednisolone base. Exemplary concentrations of PSP include 2% w/v or less, 1% w/v or less, or 0.5% w/v or less. This drug is used herein in the following descriptions by way of example. However, any of the foregoing or, indeed, any other bitter-tasting drug may be formulated using the teachings provided with PSP as an example. A contemplated composition is stable at room temperature, and may have at least a pleasant taste when administered orally.

The PSP is dissolved or dispersed in an aqueous medium. That is, the composition of drug and ingredients other than the flavorant, even if not forming a true solution, is not cloudy or milky in the aqueous medium. It is presently not known if the aqueous medium containing the drug and other ingredients is a true solution or a non-settling dispersion, but the composition containing its various constituents discussed hereinafter is preferably transparent as would be a true solution or a colloidal dispersion.

A contemplated pharmaceutical composition may be essentially free of ethanol (ethyl alcohol). Ethanol is often used in aqueous medicinal compositions as a solvent for the active medicament. However, because of its potential effects upon children and the elderly, ethanol is not utilized in a preferred composition, or, if used, is present in an amount of about ten percent by volume or less. Preferably, the ethanol, if present, is present at less than five percent.

A contemplated composition is referred to as having an aqueous medium in that water is present as a major liquid ingredient. Water is a major liquid component of a contemplated composition, but water does not constitute a majority of the composition.

In preferred embodiments, a pharmaceutically effective amount of prednisolone sodium phosphate (PSP) is present in the contemplated composition as the active ingredient. A “pharmaceutically effective amount of” PSP, or any drug, is the concentration of the drug at which a desired volume of the composition includes a therapeutic amount of the medicament. A contemplated amount of PSP can differ where compositions formulated for children and adults are contemplated, as well as when different amount of composition are contemplated for administration.

Therapeutic dosages of PSP and other contemplated drugs are well-known and are available from the above-noted texts as well as from the Physicians' Desk Reference, Medical Economics Company, Inc., Oradell, N.J., Goodman and Gilman's The Pharmacological Basis of Therapeutics, 8th ed., Gilman et al. ads., McGraw Hill, Inc., New York, N.Y. (1993), Facts and Comparisons, ASHP Hospital Formulary, Remington's, and the like.

Exemplary amounts of active PSP are present at about 0.1% to about 2% w/v. Preferably, PSP is present in an amount of less than about 0.5% w/v of the contemplated composition.

There are several standard ways in which to represent the composition makeup of a formulation. Both percent weight/volume (% w/v) and percent weight/weight (% w/w) are used frequently by those of skill in the art. Herein, the measurements presented are in units of % w/v, unless otherwise indicated. Percent weight/volume is defined to be the weight in grams of a component expressed as if the total volume of the formulation was 100 ml (i.e., number of grams per 100 ml). Percent weight/weight, however, is the weight in grams of a component expressed as if the total weight of the formulation was 100 grams (i.e., number of grams per 100 grams).

A contemplated composition also contains about 60% to about 75% and more preferably about 60 to about 70% w/v (as solids or non-volatile liquids) of a C₃-C₆ polyol. As used herein, C₃-C₆ polyol refers to the number of carbons in the basic polyol unit. “C₃-C₆ polyol” is meant to include multiple such C₃-C₆ polyol units linked together into an oligmer. For example, sucrose is a disaccharide that is comprised of two C₆ polyols linked together. In the compositions contemplated herein, at least 55% w/v, and preferably about 60% w/v, of the total pharmaceutical composition is a non-reducing disaccharide, trisaccharide, or tetrasaccharide. The term “non-reducing” as used herein refers to a saccharide (e.g., a disaccharide, trisaccharide or tetrasaccharide) that does not reduce Fehling's solution. Exemplary non-reducing polyols used in the compositions contemplated herein, include, but are not limited to sucrose, trehalose, melezitose, planteose, and raffinose. In particularly preferred compositions, the non-reducing polyol is sucrose.

No other single polyol, or mixture of polyols, other than the non-reducing polyols mentioned above, has been found to be useful in substituting for a non-reducing sugar in the recited amount in a contemplated composition. Sucrose is an exemplary and preferred non-reducing sugar. Sucrose, for example, in the recited amount provides a benefit to both taste and protection of PSP from degradation. Indeed, fructose, the C₃-C₆ polyol preferred in a PSP composition of U.S. Pat. No. 5,763,449 and No. 5,962,461, caused a composition stored at room temperature to become more acidic on aging, with concomitant decomposition of PSP. Sucrose used as described herein throughout does not have this adverse effect on the composition.

Without wishing to be bound by theory, it is believed that the substantially higher concentration of total C₃-C₆ polyol and the significant minimum amount of non-reducing disaccharide, trisaccharide, or tetrasaccharide such as sucrose required herein compared to the total C₃-C₆ polyol contemplated in a PSP composition of U.S. Pat. No. 5,763,449 or No. 5,962,461 are a major contributing factor to solving the problems related to PSP stability and taste. The minimum amount of non-reducing sugar required in the formulations is greater than the maximum amount of C₃-C₆ polyol permitted in either of the above patents. This increase in required C₃-C₆ polyol, and more specifically, in the higher percentage of non-reducing saccharide, lends to the increased stability of the active ingredient at ambient temperatures, allowing for shelf-stability of the formulation.

Again, without being bound by theory, a further hypothesis for the stability of the exemplified compositions is outlined below. The addition of sucrose in combination with the preservatives (surfactants) may stabilize PSP through the ordering of molecules. One possible explanation is that the molecules are becoming ordered due to preferred alignments around polar and non-polar regions of the molecule. This ordering of molecules are facilitated by the presence of surfactants. Surfactants contain regions of polar groups and regions of nonpolar groups and serve to bridge the interaction between immiscible or poorly miscible components (oil and water, for example). Glycyrrhetic acid or a derivative thereof contains a steroid-like multi-ring region (nonpolar, hydrophobic) connected to a sugar-like region (polar, hydrophilic), and can be represented as A-B where A represents the nonpolar region of the molecule and B represents the polar (water-loving) region of the molecule. Similarly, PSP is relatively nonpolar and can be represented as (A) while sucrose is very polar and also represented as (B). As more molecules are added, they align in a manner whereby the like molecules (A or B type) will align next to each other in a preferential manner. Based on the structure and stacking of the molecules, and the type and amount of surfactants present in the solution, one of two types of ordered structures will occur.

The first ordered structure will resemble a micelle or sphere-like structure. As the continuous phase on the solution is nonpolar, a sphere will form with all the sugar regions (B) being on the outside of the sphere and the ring structures (A) pointing to the inside of the sphere. This structure forms spontaneously in an effort to find the lowest energy state for the ordered structure. This description would be representative of oil droplets in water. If the continuous phase were polar, then the ring structures (A) would be on the outside of the sphere with the sugar regions (B) remaining on the inside of the sphere. This description would be representative of water droplets in oil. Micelles (or solid spheres) occur when the molecular structures are wedge or pie-shaped, thereby facilitating a sphere-like structure.

The second structure occurs when the molecules are roughly the same size and align more are sticks in a row, rather than wedges in a pie. Again, in an effort to minimize contact of an unfavored molecule with the continuous phase (in an effort to find the lowest energy state), the molecules will form a sheet of A-B or B-A with similar regions near each other. However, in this second situation, a bilayer will form whereby a sheet will be aligned with another sheet in a common molecular manner. An example of this would be a bilayer comprised of A-B:B-A or B-A:A-B, the alignment being dictated by the polarity of the continuous phase. In the present invention, the continuous phase is polar (B) so a B-A:A-B structure would be preferred. This ordered bilayer may remain as a sheet, or as a multi-stacked sheet or may form a liposome-like structure whereby a hollow sphere will form with the continuous phase being contained both inside and outside of the sphere.

It is important to note that these ordered structures form spontaneously and result in shielding one molecule from the continuous phase. In the present invention, PSP would be either contained within the core of the micelle or within the middle of the bilayer with minimal to no contact with the continuous (sucrose solution) phase. This is significant as PSP degrades in water that would be present in the continuous phase. Ordered structures would form to specifically exclude water coming in contact with PSP. The result of such an ordered structure excluding water from contacting PSP is that the source of degradation would be removed and the PSP formulation would be imparted with an unexpected stability. The formation of micelles and bilayer/liposome ordered structures is well known and they are often used in an effort to protect a molecule from the environment present in the continuous phase.

In addition to the C₃-C₆ non-reducing polyols described above, exemplary other C₃-C₆ polyols that can be present in a contemplated composition include propylene glycol, glycerin (glycerol), threose, threitol, erythrose, erythritol, ribose, arabinose, lyxose, maltitol, sorbitol, sorbose, glucose, mannose, mannitol, levulose, dextrose, maltose, fructose, xylitol, inositol, galactose, xylose, fructose, sucrose, 1,2,6-hexanetriol and the like. However, the presence of one or more reducing sugars such as fructose, maltose or galactose is not preferred as those polyols oxidize more readily than do non-reducing sugars, and that oxidation is believed to lead to acidification of the composition and decomposition of the PSP. Maltitol is another molecule containing two linked C₆ polyols that is deemed therefore to be a C₆ polyol. A C₃-C₆ polyol serves several functions in the compositions of the invention, including acting as a solvent for the system, contributing to the stability of the active compound at room temperature, as well as being a masking agent for bitter flavors.

One or more C₆ reducing sugar oligosaccharide polyols such as sorbitol USP, maltitol NF at 75 percent solids, liquid fructose (an α-hydroxyketose that is a reducing sugar) such as that available under the mark KRYSTAR™, from A.E. Staley Mfg. Co., Decatur, Ill. that is sold as an aqueous liquid, about 77 weight percent of which is fructose, as well as sugars such as glucose, xylitol and the like can also be present.

In one preferred embodiment, a mixture of two or more C₃-C₆ polyols is utilized. Such a mixture utilizes a C₃ polyol such as glycerin or 1,3-propanediol (propylene glycol), with both of those C₃ polyols being preferred for use.

This C₃ and C₆ polyol mixture, when utilized, is typically present at a weight ratio of 1:6 C₃:C₆ polyol to about 1:70 C₃:C₆ polyol, as non-volatiles, provided that the total polyol weight percentage in the composition is between 55% and 75%. In another preferred embodiment, polyols other than a C₆ polyol (i.e. C₃, C₄, and C₅ polyols) constitute less than about 5 weight percent of the total composition, in certain preferred embodiments, less than about 2%.

In addition to the water, the PSP and the C₃-C₆ polyol, the composition of the invention also may comprise about 3% to about 10% w/v polyvinylpyrrolidone (PVP), and more preferably about 3% to about 7% w/v PVP. PVP (commercially (BASF) known as Povodone) is commercially available from a number of suppliers under a number of designations with molecular weights ranging from 10,000 to 1,500,000 Da. The PVPs sold under the Trademark KOLLIDON® K25, K30 and K90 having weight-average molecular weights of 28,000-34,000, 44,000-54,000 and 1,000,000-1,500,000, respectively, are preferred for use here, with the K25 and K30 being most preferred.

PVP is dissolved or dispersed in the water of the aqueous medium and serves to assist in dissolving or dispersing the bitter-tasting drug in that medium, as well as stabilizing the compound and masking the flavor of the bitter-tasting drug. The disclosures of Volker Bühler's book, Kollidon, BASF Aktiengesellshaft, Ludwigshafen, Germany (1992) teach the use of PVP as both a solubilization aid for several drugs as well as for masking the bitter taste of acetaminophen. An exemplary formulation for an oral PVP- and acetaminophen-containing composition is provided at page 113, Table 81 of the above Bühler text. PVP has been used as a thickening agent (U.S. Pat. No. 5,496,541), texture-modifier (US 2003/0161888), and binder (U.S. Pat. No. 4,966,770) and as the primary constituent in tablet coatings. Other agents can be used as a thickener, texture modifier or binder similar to PVP and include but are not limited to colloidal cellulose, hydrated silica, polyethylene glycol, sodium carboxymethylcellulose, hydroxyethylcellulose, hydroxypropylcellulose, guar gum or xanthan gum, and hydroxypropyl methylcellulose.

In one aspect of the invention, there is a liquid pharmaceutical composition comprising a pharmaceutically effective amount of a drug dissolved or dispersed in an aqueous medium that is preferably free of ethanol, said aqueous medium comprising water, about 3% to about 10% w/v polyvinylpyrrolidone (PVP), about 60% to about 75% w/v of C₃-C₆ polyol and including more than 55% w/v of the total composition of a non-reducing disaccharide, trisaccharide, or tetrasaccharide wherein the composition is stable at room temperature.

The use of the C₃-C₆ polyols with the PVP are effective in stabilizing the active ingredient in a room temperature formulation. Therefore, what is contemplated is a combined formulation of polyols, PVP and an active ingredient that is stable at room temperature as an aqueous solution. While some embodiments have preferred compositions that are pleasant-tasting, other preferred compositions need not be pleasant-tasting because it is a significant advantage of the present invention that it is stable at room temperature and provided in a liquid form.

The polyols and PVP, while stabilizing the composition, are not necessarily sufficient to mask the bitter taste of a steroidal drug such as PSP. This fact remains even when further sweeteners such as sodium saccharin USP present at 0.05-2% w/v or aspartame present at about 0.1 to about 2% w/v and further flavorants are admixed with the composition. A further debittering agent is still required to be present in order to create a pleasant tasting formulation.

That further debittering agent is found to be glycyrrhetic acid, derivatives of glycyrrhetic acid such as esters, amides, or thioesters, or salts of either the glycyrrhetic acid or glycyrrhizinate derivatives that can be present at about 0.01% to about 5.0% w/v, or, more preferably, for example, about 0.01% to about 0.5% w/v, or about 1.0% to about 5.0% w/v. Other preferred ranges include about 0.01% to about 0.05% w/v, or about 0.1% to about 0.5.0% w/v AG. Preferably, glycyrrhetic acid and derivatives thereof are a 10% solids solution, such as for example, ammonium glycyrrhizinate is a 10% solids solution in glycerol or propylene glycol under the name MAGNASWEET® MM 110 or MM 115 which is added in the amount of about 0.5 to about 5.0% w/v. Ammonium glycyrrhizinate (AG) is a preferred salt for use as a debittering agent. The glycyrrhetic acid, deriviative or salt is preferably present at a weight ratio as compared to the PSP or other drug of about 1:100 to about 1:5, more preferably about 1:50 to about 1:10, and most preferably at weight ratio of about 1:20 the glycyrrhizinate to drug. Also contemplated are weight ratios of glycerrhizinate to the PSP of 1:100, 1:95, 1:90, 1:85, 1:80, 1:75, 1:70, 1:65, 1:60, 1:55, 1:50, 1:45, 1:40. 1:35, 1:30, 1:25, 1:20, 1:15, 1:10, and 1:5.

In another aspect of the invention, therefore, there is provided a composition that includes a pharmaceutically effective amount of a drug dissolved or dispersed in an aqueous medium that is preferably low in ethanol (e.g. less than 10%) or optionally free of ethanol, said aqueous medium comprising water, about 3% to about 10% w/v polyvinylpyrrolidone (PVP) as an optional component, about 55% to about 75% w/v of C₃-C₆ polyol, in which more than 55% w/v of the total composition is a non-reducing disaccharide, trisaccharide, or tetrasaccharide, about 0.01% to about 5.0% w/v glycyrrhetic acid, derivative or salt thereof, and one or more flavorants, that is stable at room temperature and may have a pleasant taste. In preferred embodiments, wherein ammonium glycyrrhizinate used herein is a 10% ammonium glycyrrhizinate solids solution in glycerol or propylene glycol. Such solutions (e.g., MAGNASWEET MM 110 or MM 115) are discussed further below.

Ammonium glycyrrhizinate is available as a 10% weight percent-solution in glycerin or propylene glycol from MacAndrews & Forbes Company of Camden, N.J. under the name MAGNASWEET® MM 110 or MM 115, and also as a white, amorphous powder as MM 150. In particularly preferred embodiments, the compositions of the invention, the compositions comprise between about 0.1 to about 5% w/v MAGNASWEET MM 110 or MM 115. Ammonium glycyrrhizinate is the monoammonium salt of a triterpenoid saponin that consists of an aglycone of glycyrrhetic acid and a sugar moiety of two glucuronic acid units linked to each other. This is said by its manufacturer to be about 50 to about 100 times sweeter than sucrose, and is known to be useful in masking bitterness. Other agents and processes have been used to provide a taste-masking or sweetening effect similar to ammonium glycyrrhizinate and include various flavors, sweeteners, acidic amino acids, lipids and surfactants including but not limited to dimethylaminoethyl methacrylate and neutral methacrylic acid esters, (WO 2004/022037 A1), processing approaches such as micro-encapsulation with resins and proteins, gelatinized starch, gums, cyclodextrins, chitosan, liposomes, removal of bitter contaminants by ion exchange resins, chemical modification and specific salt preparation.

Although ammonium glycyrrhizinate is a known bitterness-masking agent, as is PVP, neither material alone or with the before-discussed sweeteners and flavorants is sufficient to mask the bitter taste of a contemplated bitter-tasting drug. Rather, PVP and ammonium glycyrrhizinate appear to potentiate each other to provide the desired bitterness-masking effect.

The mechanism by which the bitterness-masking is achieved is unknown. In U.S. Pat. No. 5,763,449 and No. 5,962,461 propose that polyvinylpyrrolidone, ammonium glycyrrhizinate and PSP may form a presently undefined complex in the aqueous medium, that complex acts to shield taste buds from the bitterness inherently present in the bitter-tasting drug.

The determination of a bitter taste is carried out by standard, well-known practices, and is a characteristic often listed along with a description of the drug in texts such as The Merck Index, 11th ed., S. Budavari et al. eds., Merck & Co., Inc., Rahway, N.J. (1989) and Remington's Pharmaceutical Sciences, 18^{th} ed, A. Gennaro ed., Mack Publishing Co., Easton, Pa. (1990).

In particular embodiments, the final pH value of the compositions of the invention is about 6 to about 8, and preferably about 6.5 to about 7.5, and more preferably about 6.7 to about 7.4, and most preferably about 6.8 to about 7.2. A system of sodium hydroxide (1 N) and hydrochloric acid (10 N) or one of citric acid and sodium citrate is typically used for pH value adjustments and maintenance. Mono- and dibasic sodium or potassium phosphate salts and other salts can also be used as a buffer system to maintain the pH value of a contemplated composition. It is preferred to include a minimal amount, e.g., less than about 5% w/v, and more preferably less than about 2% w/v, of acid and base as buffering salts such as the above mono- and dibasic phosphate salts, because too much salt present in the composition can result in an unpleasant taste being imparted to the composition. Such high concentrations are typically not needed because of the preferred absence of reducing sugars that produce acid groups upon oxidation.

As was noted previously, a composition of the invention may also contain additional sweeteners, and flavorants, as well as colorants and thickeners. Flavorants such as bubble gum and chocolate flavors can provide opacity or translucency to a contemplated composition, while the composition other than the flavorant is transparent. Exemplary thickeners include sodium alginate, gelatin or a polyalkylene oxide such as the polyoxyethylene-polyoxypropylene-polyethylene terpolymer available under the name PLURONIC® F68 having an average of 75 polymerized ethylene oxide units on either side of 30 polymerized propylene oxide units, F-87 having 62 polymerized ethylene oxide units on either side of 39 polymerized propylene oxide units, or F-88 having an average of about 97 polymerized ethylene oxide groups on either side of about 39 polymerized propylene oxide groups that are available from BASF, Mount Olive, N.J. A contemplated aqueous liquid pharmaceutical composition has a viscosity at 25° C. between that of water and about that of corn syrup at 25° C.

It has been found that a contemplated composition for room temperature storage is unexpectedly difficult to protect from microbial attack while maintaining the activity of the PSP. By comparison, a PSP composition of U.S. Pat. No. 5,763,449 and No. 5,962,461 (each incorporated herein by reference in its entirety) contained ethanol and had to be refrigerated to provide the desired PSP stability and freedom from microbial attack.

Conventional preservatives such as sodium benzoate NF, methylparaben NF and propylparaben NF as discussed in U.S. Pat. No. 5,763,449 and No. 5,962,461 also may be provided in the compositions of the present invention. Inclusion of such preservatives and removal of PVP from a test composition showed that protection from microbial attack was present, indicating that PVP may be binding to, and thereby partially removing, one or more of those preservatives from effective use in a contemplated composition.

Those conventional preservatives, while useful, were ineffective in providing the desired room temperature freedom from microbial attack for a composition contemplated here. Indeed, Remington's Pharmaceutical Sciences, 18th ed., A. Gennaro ed., Mack Publishing Co., Easton, Pa. (1990) at page 1173 notes that sodium benzoate, used herein is not effective at a pH value greater than about 4. To the contrary, however, removal of the benzoate from a composition of the invention led to increased microbial attack, so it is concluded that this preservative is, in fact, useful at the higher pH values utilized herein.

It has been further been found that inclusion of sodium or potassium sorbate with the above preservatives along with the above-recited amount of C₃-C₆ polyol can provide the desired room temperature stability of PSP and a desired level of freedom from microbial attack. Benzyl alcohol can also be present as a preservative. An effective amount of sodium or potassium sorbate or benzyl alcohol is the amount at which microbial attack is diminished in comparison to a similarly constructed composition, less the preservative. Methods for evaluating the efficacy of preservatives in pharmaceutical formulations are known to those skilled in the art. Each of the preservatives is utilized in an effective amount that is less than 1% w/v of the composition, and more typically at about 0.01% to about 0.75% w/v.

Composition Preparations

A contemplated pleasant-tasting aqueous liquid pharmaceutical composition is readily prepared using pharmaceutical formulations techniques known to those of skill in the art. Thus, in an exemplary procedure where a C₃ polyol is utilized, a solution or dispersion of about 30% w/v PVP is prepared in water (composition A). About one part PSP is slurried with about 5 parts by weight C₃ polyol (glycerin or propylene glycol or both) (slurry being composition B). The two compositions, composition A and composition B, are admixed and heated to a temperature of about 45° C. with continued agitation. Agitation is continued at that temperature until a clear, non-settling solution or dispersion is formed, which generally takes about 30 minutes. Where less than about 5% w/v C₃ polyol is used, as is employed in certain preferred embodiments herein, the PSP is admixed directly with the aqueous PVP.

The aqueous composition formed in the above process is cooled at a temperature below about 30° C. and the ammonium glycyrrhizinate, other C₃-C₆ polyols, flavorants, colorant if used and remaining ingredients are admixed until a homogenous composition is obtained. These additions are typically carried out serially, with admixture to homogeneity between each admixture. The preservatives are added and the pH value is thereafter adjusted as required. The examples that follow illustrate these procedures more fully.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

### Comparative Example: U.S. Pat. No. 5,962,461 Ethanol-Containing Prednisolone Sodium Phosphate Oral Liquid

Two pleasant-tasting liquids for oral administration containing prednisolone sodium phosphate as active ingredient were prepared containing the ingredients and amounts shown in the table below for liquid 1 and liquid 2.

| | | Liquid 1 | Liquid 2 |
|---|---|---|---|
| | Ingredient and Number | (w/v or w/w %) | (w/v or w/w %) |
| 1. | Prednisolone Sodium | 0.134 | 0.403 |
| | Phosphate USP | | |
| 2. | Polyvinylpyrrolidone, | 5.0 | 5.0 |
| | USP (PVP; K25) | | |
| 3. | Ethanol, USP | 1.71 | 1.71 |
| 4. | Purified Water, USP | 20.0 | 20.0 |
| 5. | Sodium Benzoate, NF | 0.15 | 0.15 |
| 6. | MAGNASWEET ™ 110 (a 10% | 2.0 | 2.0 |
| | solution of ammonium | | |
| | glycyrrhizinate)¹ | | |
| 7. | Sorbitol Solution, USP | 10.0 | 10.0 |
| | (70% solids) | | |
| 8. | Sodium Hydroxide, USP | q.s. | q.s. |
| | (1 N) | | |
| 9. | Citric Acid, USP | q.s. | q.s. |
| | (50%) | | |
| 10. | Maltitol Solution, NF | q.s. | 20.00 |
| | (75% solids) | | |
| 11. | Flavorant | 0.65 | .75 |
| 12. | Liquid Fructose | — | q.s. |
| | (77.0-77.5% solids) | | |

¹A 10% solids solution in glycerin or propylene glycol from MacAndrews & Forbes Co.

Purified water, USP was charged into a kettle and agitation of the water begun. The Povidone 25, USP was slowly added to the mixing water and the resulting admixture agitated until all of the Povidone 25, USP was dissolved to form phase one.

Further purified water, USP was added to a separate vessel and stirring of the water begun. To the stirring solution was added prednisolone sodium phosphate, USP, and stirring was continued until all particles were dissolved to form phase two.

Still further purified water, USP was added to a third vessel and mixing of the water began. Sodium benzoate, NF was admixed with agitation until all particles were dissolved to form phase three.

Phase one, phase two and phase three were admixed together with agitation, followed by admixture of Sorbitol Solution 70% USP, MAGNASWEET® (contains a 10% solution of ammonium glycyrrhizinate), ethanol, flavorant, Maltitol solution NF, and with a sufficient amount of liquid fructose is added to reach the final volume of the formulation. Agitation was continued to ensure homogeneity. The pH value was measured and adjusted to 7.0 (±0.3) with citric acid or sodium hydroxide as needed.

After storage of above Liquid 2 at a temperature of 40° C. and 75% relative humidity (RH) for a time period of three months, the composition changed from a clear yellow color to a clear yellow-orange and then a clear amber color, the pH value dropped from 6.9 to 5.4 and the percentage of prednisolone sodium phosphate dropped from 102 percent to 83 percent. Similar samples stored at 30° C. and 60% RH or at 25° C. and 60% RH exhibited no color changes, a drop of less than one pH unit and a 6% or no loss of prednisolone sodium phosphate over the same time period. Those results suggest a temperature-dependent acidification that leads to the decomposition of the prednisolone sodium phosphate.

### EXAMPLE 2

### Room Temperature Storage Stable Pleasant Tasting Aqueous Composition of Sodium Prednisolone Phosphate

An aqueous, room temperature storage stable and pleasant tasting PSP composition containing the following amounts of ingredients were prepared and subjected to 25° C. and 60% RH storage for a period of nine months with a good product stability profile. The composition was also placed under accelerated storage conditions at a temperature of 40° C. and 75% RH for a period of three months, a recognized storage condition which simulates a two years of shelf life at room temperature.

Room Temperature Storage Stable Pleasant Tasting PSP
Composition, Presented in Both % w/w and % w/v

| | | Liquid | Liquid |
|---|---|---|---|
| | Ingredient and Number | (% w/v) | (% w/w) |
| 1. | Prednisolone Sodium | 0.40 | 0.32 |
| | Phosphate, USP | | |
| 2. | Sucrose NF | 60.0 | 47.28 |
| 3. | Polyyvinylpyrrolidone, | 5.00 | 3.94 |
| | USP (PVP; K25) | | |
| 4. | Propylene glycol, USP | 1.00 | 0.79 |
| 5. | Sorbitol Solution, | 5.00 | 2.76 |
| | (70% solids), USP | | |
| 6. | MAGNASWEET ® 110 (a 10% | 2.00 | 1.58 |
| | solution of ammonium | | |
| | glycyrrhizinate) | | |
| 7. | Flavorant (Grape) | 0.75 | 0.59 |
| 8. | Sodium Benzoate, NF | 0.15 | 0.12 |
| 9. | Methylparaben, NF | 0.10 | 0.08 |
| 10. | Propylparaben, NF | 0.05 | 0.04 |
| 11. | Potassium Sorbate, NF | 0.50 | 0.39 |
| 12. | EDTA Disodium Dihydrate, USP | 0.05 | 0.04 |
| 13. | Sodium Hydroxide, USP | q.s., | q.s., |
| | (1 N) | pH 7.0 | pH 7.0 |
| 14. | Hydrochloric Acid, USP | q.s., | q.s., |
| | (1 N) | pH 7.0 | pH 7.0 |
| 15. | Dibasic Sodium Phosphate, | 1.45 | 1.14 |
| | Anhydrous, USP | | |
| 16. | Monobasic Sodium Phosphate, | 0.16 | 0.13 |
| | Monohydrate, USP | | |
| 17. | Purified Water, USP | 23.39 | 40.82 |

A 6,000 liter contemplated aqueous composition of PSP was prepared by the admixture of three substantially homogeneous sub-compositions. The first sub-composition was prepared by admixing the propylene glycol (4), methylparaben (9), propylparaben (10) and grape flavor (7) until substantially homogeneous. The second subcomposition was prepared from a first portion of purified water (17), about 37 percent of the total compostion's weight, was heated to about 65° C.±about 5° C. and then admixed with the sucrose (2), soribitol (5), and MAGNASWEET® 110 (6) until substantially homogeneous. The povidone (3) is then added to the batch tank and then mixed until completely wetted and dispersed. The resulting admixture was cooled to a temperature of about 40° C. or below. The third sub-composition was prepared by admixture and agitation to substantial homogeneity of a second portion of purified water that constituted about 15 percent of the total composition comprising prednisolone sodium phosphate (1), to which was added sequentially the mono- and dibasic sodium phosphates (16 and 15), EDTA (12), sodium benzoate (8), and potassium sorbate (11).

The second and third sub-compositions were admixed to substantial homogeneity, followed by admixture of the first sub-composition and further mixing to substantial homogeneity. The pH value was adjusted to the desired value of about 7.0 (6.9-7.3), and remainder of the purified water (15), about 4 percent, was then admixed to complete the preparation of the desired composition in a final volume of 6,000 liters. It is to be understood that such a desired composition can also be prepared in other manners.

Illustrative lots submitted for microbiological testing pursuant to the current USP 26 for both (a) Preservative Effectiveness Test (PET), Chapter <51>, and (b) Microbial Limits, Chapter <61>, as well as for analytical Assay Determination under the current USP 26, Validation Compendia, Chapter <1225>, all passed their respective testing. Following acceptable results from the in-process testing, the batch is approved for packaging. The large-scale production at a 6,000-liter scale results in approximately 24,200 8-oz bottles.

Each of the patents and articles cited herein is incorporated by reference. The use of the article “a” or “an” is intended to include one or more.

The foregoing description and the examples are intended as illustrative and are not to be taken as limiting. Still other variations within the spirit and scope of this invention are possible and will readily present themselves to those skilled in the art.

What is claimed is:

1. A liquid pharmaceutical composition comprising a pharmaceutically effective amount of a drug dissolved or dispersed in an aqueous medium, said aqueous medium comprising:
water;
about 3% to about 10% w/v polyvinylpyrrolidone (PVP);
about 55% to about 75% w/v of C₃-C₆ polyol, wherein at least 55% w/v of the total composition is a non-reducing disaccharide, trisaccharide, or tetrasaccharide;
about 0.01% to about 0.5% w/v of a glycyrrhetic acid, glycyrrhizinate derivative, or salt thereof; and
one or more flavorants, said composition being stable at room temperature.

2. The liquid pharmaceutical composition according to claim 1 wherein the drug is a steroid.

3. The liquid pharmaceutical composition according to claim 1 wherein the drug is selected from the group consisting of acetaminophen, terfenadine, guaifenesin, dexamethasone, triamcinolone, trimethoprim, prednisolone, ibuprofen, prednisolone sodium phosphate, methacholine, neostigmine, epinephrine, albuterol, pseudoephedrine hydrochloride, diphenhydramine, chlorpheniramine maleate, phenothiazine, chlorpromazine, chlordiazepoxide, amitriptyline, barbiturates, diphenylhydantoin, caffeine, morphine, demerol, codeine, iphenoxylate, opiate derivatives, benzodiazepines, prednisone, lidocaine, salicylic acid, sulfonamides, chloroquine, pregnanes, vitamin preparations, minerals and penicillins.

4. The liquid pharmaceutical composition according to claim 1 wherein the drug is prednisolone sodium phosphate.

5. The liquid pharmaceutical composition according to claim 1, wherein the glycyrrhetic derivative is ammonium glycyrrhizinate.

6. The liquid pharmaceutical composition according to claim 1 wherein said drug is present in an amount of less than about 1.0% w/v of the total composition.

7. The liquid pharmaceutical composition according to claim 1 wherein said PVP is present at about 3 to about 7% w/v of the total composition.

8. The liquid pharmaceutical composition according to claim I wherein said PVP is present at about 5% w/v of the total composition.

9. The liquid pharmaceutical composition according to claim 1 wherein said C₃-C₆ polyol is present as a mixture of one or more C₃ polyols and one or more C₆ polyols.

10. The liquid pharmaceutical composition according to claim 9 wherein said mixture of C₃ polyols and C₆ polyols is present in an amount of about 60 to about 75% w/v.

11. The liquid pharmaceutical composition according to claim 9 wherein the weight ratio of said C₃ polyols to said C₆ polyols is about 1:6 to about 1:70.

12. The liquid pharmaceutical composition according to claim 9 wherein the weight ratio of said C₃ polyols to said C₆ polyols is about 1:25.

13. The liquid pharmaceutical composition according to claim 1 wherein the non-reducing disaccharide or trisaccharide is present in about 60% w/v of the total composition.

14. The liquid pharmaceutical composition according to claim 1 wherein the non-reducing disaccharide, trisaccharide, or tetrasaccharide is selected from the group consisting of sucrose, trehalose, melezitose , planteose, and raffinose.

15. The liquid pharmaceutical composition according to claim 1 wherein the non-reducing disaccharide is sucrose.

16. The liquid pharmaceutical composition according to claim 1 that further includes one or both of (i) sodium or potassium sorbate and (ii) benzyl alcohol in an amount sufficient to act as a preservative.

17. The liquid pharmaceutical composition according to claim 1 that further includes dissolved buffer salts that provide a buffered pH value of about 6 to about 8.

18. A liquid pharmaceutical composition comprising a pharmaceutically effective amount of prednisolone sodium phosphate present in an amount of less than about 1.0% w/v dissolved or dispersed in an aqueous medium buffered at a pH value of about 6 to about 8, said aqueous medium comprising water, buffer salts, about 3% to about 10% w/v PVP, a mixture of one or more C₃ polyols and one or more C₆ polyol that is present in an amount of about 55% to about 75% w/v in which the weight ratio of said C₃ polyol to said C₆ polyol is about 1:6 to about 1:70, said mixture of C₃ polyol and C₆ polyol includes more than 55% w/v of the total composition of sucrose, about 0.01% to about 0.5% w/v of glycyrrhetic acid, derivative or salt thereof, and one or more flavorants, said composition being stable at room temperature and having a pleasant taste.

19. The liquid pharmaceutical composition according to claim 18, wherein the glycyrrhetic acid, derivative or salt is ammonium glycyrrhizinate.

20. The liquid pharmaceutical composition according to claim 18 that further includes one or both of (i) sodium or potassium sorbate and (ii) benzyl alcohol present at less than 1 weight percent of the composition.

21. The liquid pharmaceutical composition according to claim 18 wherein said one or both of (i) sodium or potassium sorbate and (ii) benzyl alcohol in an amount of about 0.01 to about 0.75% w/v.
